SEQUENCE LISTING (12 sequences)

SEQ ID NO: 1 - protein, 203 aa, synthetic construct
QRQGRMLTFEAAAKKDDWFVPAEAAAKFSHAAGIAYEAAAKLNIAGIWKWEAAAKEVIKEAVDYARSGNEAAAKLTNKPKEDYFPMEAAAKNSKKLEKGYEAAAKRTTPSVVAFEAAAKEISAKILAYLKEYEAAAKKAVITVPAYFEAAAKVYDLGGGTFEAAAKSVSTVSLPFEAAAKLIDRTRKPIEAAAKRSEFEKMTA

SEQ ID NO: 2 - DNA, 612 bp, synthetic construct
  source 1..612: mol_type = other DNA
cagcgccagggccgcatgctgacctttgaagcggcggcgaaaaaagatgattggtttgtgccggcggaagcggcggcgaaatttagccatgcggcgggcattgcgtatgaagcggcggcgaaactgaacattgcgggcatttggaaatgggaagcggcggcgaaagaagtgattaaagaagcggtggattatgcgcgcagcggcaacgaagcggcggcgaaactgaccaacaaaccgaaagaagattattttccgatggaagcggcggcgaaaaacagcaaaaaactggaaaaaggctatgaagcggcggcgaaacgcaccaccccgagcgtggtggcgtttgaagcggcggcgaaagaaattagcgcgaaaattctggcgtatctgaaagaatatgaagcggcggcgaaaaaagcggtgattaccgtgccggcgtattttgaagcggcggcgaaagtgtatgatctgggcggcggcacctttgaagcggcggcgaaaagcgtgagcaccgtgagcctgccgtttgaagcggcggcgaaactgattgatcgcacccgcaaaccgattgaagcggcggcgaaacgcagcgaatttgaaaaaatgaccgcgtaa

SEQ ID NO: 3 - protein, 69 aa, synthetic construct
QRQGRMLTFEAAAKKDDWFVPAEAAAKFSHAAGIAYEAAAKLNIAGIWKWEAAAKEVIKEAVDYARSGN

SEQ ID NO: 4 - DNA, 210 bp, synthetic construct
  source 1..210: mol_type = other DNA
cagcgccagggccgcatgctgacctttgaagcggcggcgaaaaaagatgattggtttgtgccggcggaagcggcggcgaaatttagccatgcggcgggcattgcgtatgaagcggcggcgaaactgaacattgcgggcatttggaaatgggaagcggcggcgaaagaagtgattaaagaagcggtggattatgcgcgcagcggcaactaa

SEQ ID NO: 5 - protein, 12 aa, synthetic construct
LTNKPKEDYFPM

SEQ ID NO: 6 - DNA, 39 bp, synthetic construct
  source 1..39: mol_type = other DNA
ctgaccaacaaaccgaaagaagattattttccgatgtaa

SEQ ID NO: 7 - protein, 112 aa, synthetic construct
NSKKLEKGYEAAAKRTTPSVVAFEAAAKEISAKILAYLKEYEAAAKKAVITVPAYFEAAAKVYDLGGGTFEAAAKSVSTVSLPFEAAAKLIDRTRKPIEAAAKRSEFEKMTA

SEQ ID NO: 8 - DNA, 339 bp, synthetic construct
  source 1..339: mol_type = other DNA
aacagcaaaaaactggaaaaaggctatgaagcggcggcgaaacgcaccaccccgagcgtggtggcgtttgaagcggcggcgaaagaaattagcgcgaaaattctggcgtatctgaaagaatatgaagcggcggcgaaaaaagcggtgattaccgtgccggcgtattttgaagcggcggcgaaagtgtatgatctgggcggcggcacctttgaagcggcggcgaaaagcgtgagcaccgtgagcctgccgtttgaagcggcggcgaaactgattgatcgcacccgcaaaccgattgaagcggcggcgaaacgcagcgaatttgaaaaaatgaccgcgtaa

SEQ ID NO: 9 - protein, 198 aa, synthetic construct
QRQGRMLTFEAAAKKDDWFVPAEAAAKFSHAAGIAYEAAAKLNIAGIWKWEAAAKEVIKEAVDYARSGNEAAAKLTNKPKEDYFPMEAAAKGIDLGTTNSEAAAKTVRANERDYEAAAKGLDKTEKEMEAAAKVLELSGGTFEAAAKLVKKIKEEYEAAAKMSRKIRQNNEAAAKKSDKMALTREAAAKASIKRLMGT

SEQ ID NO: 10 - DNA, 597 bp, synthetic construct
  source 1..597: mol_type = other DNA
cagcgccagggccgcatgctgacctttgaagcggcggcgaaaaaagatgattggtttgtgccggcggaagcggcggcgaaatttagccatgcggcgggcattgcgtatgaagcggcggcgaaactgaacattgcgggcatttggaaatgggaagcggcggcgaaagaagtgattaaagaagcggtggattatgcgcgcagcggcaacgaagcggcggcgaaactgaccaacaaaccgaaagaagattattttccgatggaagcggcggcgaaaggcattgatctgggcaccaccaacagcgaagcggcggcgaaaaccgtgcgcgcgaacgaacgcgattatgaagcggcggcgaaaggcctggataaaaccgaaaaagaaatggaagcggcggcgaaagtgctggaactgagcggcggcacctttgaagcggcggcgaaactggtgaaaaaaattaaagaagaatatgaagcggcggcgaaaatgagccgcaaaattcgccagaacaacgaagcggcggcgaaaaaaagcgataaaatggcgctgacccgcgaagcggcggcgaaagcgagcattaaacgcctgatgggcacctaa

SEQ ID NO: 11 - protein, 168 aa, synthetic construct
KHYTYNFDFDYAAYAAYQVDGYFIGLAAYAAYKIFDNNLNFAAYAAYKLTFLISFYAAYAAYLADLLLAFYAAYAAYWKEITTAEEAAYAAYFKLVIPDNKAAYAAYNKKDKNPKDAAYAAYKLTNKHSPEAAYAAYQNIKLKPLKFNTEAAYAAYEKIFEIRFETND

SEQ ID NO: 12 - DNA, 507 bp, synthetic construct
  source 1..507: mol_type = other DNA
aaacattatacctataactttgattttgattatgcggcgtatgcggcgtatcaggtggatggctattttattggcctggcggcgtatgcggcgtataaaatttttgataacaacctgaactttgcggcgtatgcggcgtataaactgacctttctgattagcttttatgcggcgtatgcggcgtatctggcggatctgctgctggcgttttatgcggcgtatgcggcgtattggaaagaaattaccaccgcggaagaagcggcgtatgcggcgtattttaaactggtgattccggataacaaagcggcgtatgcggcgtataacaaaaaagataaaaacccgaaagatgcggcgtatgcggcgtataaactgaccaacaaacatagcccggaagcggcgtatgcggcgtatcagaacattaaactgaaaccgctgaaatttaacaccgaagcggcgtatgcggcgtatgaaaaaatttttgaaattcgctttgaaaccaacgattaa

(19) United States
(12) Patent Application Publication
(10) Pub. No.: US 2025/0360188 A1
(43) Pub. Date: Nov. 27, 2025

(54) MYCOPLASMA HYOPNEUMONIAE SUBUNIT VACCINE AND PREPARATION METHOD AND USE THEREOF

(71) Applicant: JIANGSU ACADEMY OF AGRICULTURAL SCIENCES, Nanjing (CN)

(72) Inventors: Yanfei YU, Nanjing (CN); Zhixin FENG, Nanjing (CN); Qiyan XIONG, Nanjing (CN); Jia WANG, Nanjing (CN); Maojun LIU, Nanjing (CN); Guoqing SHAO, Nanjing (CN); Yanna WEI, Nanjing (CN); Ting YUAN, Nanjing (CN); Jiahao LI, Nanjing (CN)

(73) Assignee: JIANGSU ACADEMY OF AGRICULTURAL SCIENCES, Nanjing (CN)

(21) Appl. No.: 19/271,842

(22) Filed: Jul. 17, 2025

Related U.S. Application Data
(63) Continuation of application No. PCT/CN2024/109821, filed on Aug. 5, 2024.

(30) Foreign Application Priority Data
May 27, 2024 (CN) 202410662261.3

(51) Int. Cl. A61K 39/02; A61K 39/00; A61P 37/04; C07K 14/35; C12P 21/02

(52) U.S. Cl. CPC A61K 39/0241; A61P 37/04; C07K 14/35; C12P 21/02; A61K 2039/55588; C07K 2319/00

(57) ABSTRACT

A Mycoplasma hyopneumoniae (Mhp) subunit vaccine and a preparation method and use thereof are provided. An antigen for the Mhp subunit vaccine is a fusion protein of Mhp, and has an amino acid sequence shown in SEQ ID NO: 1. The preparation method includes the following steps: (1) preparing an aqueous phase including the fusion protein; and (2) mixing an oil phase with the aqueous phase according to a volume ratio of 1:(0.8-1.2), and emulsifying to produce the vaccine. The Mhp subunit vaccine can effectively protect pigs in the long term, which provides an effective means for the prevention and control of mycoplasma pneumonia of swine.

### CROSS REFERENCE TO THE RELATED APPLICATIONS

This application is a continuation application of International Application No. PCT/CN2024/109821, filed on Aug. 5, 2024, which is based upon and claims priority to Chinese Patent Application No. 202410662261.3, filed on May 27, 2024, the entire contents of which are incorporated herein by reference.

### SEQUENCE LISTING

The instant application contains a Sequence Listing which has been submitted in XML format via EFS-Web and is hereby incorporated by reference in its entirety. Said XML copy is named GBHS016-PKG_Sequence_Listing.xml, created on May 14, 2025, and is 14,271 bytes in size.

### TECHNICAL FIELD

The present disclosure belongs to the field of animal vaccines, and specifically relates to a Mycoplasma hyopneumoniae (Mhp) subunit vaccine and a preparation method and use thereof.

### BACKGROUND

Mhp is the major pathogen causing mycoplasma pneumonia of swine. Affected pigs primarily show symptoms such as coughing, dyspnea, emaciation, and growth retardation. Mhp often triggers the secondary and mixed infections of other pathogens, such as porcine circovirus (PCV), porcine reproductive and respiratory syndrome virus (PRRSV), and Streptococcus suis (SS), so as to cause porcine respiratory disease complex, resulting in economic losses.

The prevention and control of mycoplasma pneumonia of swine is mainly based on measures such as antibacterial drugs and vaccine immunization. However, antibiotics are prone to drug resistance. The relapse easily occurs once the medication is stopped. Vaccines remain an important means for controlling mycoplasma pneumonia of swine. The current vaccines for mycoplasma pneumonia of swine mainly include inactivated vaccines and attenuated vaccines. The inactivated vaccines dominate the market. However, the inactivated vaccines can only provide partial protection and show limited effects on preventing the infection and the spread of pathogens. The attenuated vaccines involve cumbersome clinical operations, and the corresponding immunization modes, such as intrapulmonary injection, have high technical requirements for operating personnel. The novel vaccines, such as recombinant subunit vaccines, have advantages such as relatively high safety and available large-scale production compared with the traditional vaccines and are a hot spot for institutional research inside and outside China. However, there are currently no subunit vaccines that can effectively protect pigs in the long term.

### SUMMARY

An objective of the present disclosure is to provide a Mhp subunit vaccine, which can effectively protect pigs in the long term. Thus, the present disclosure provides an effective means for the prevention and control of mycoplasma pneumonia of swine.

The objective of the present disclosure is achieved through the following technical solutions:

The present disclosure provides a Mhp subunit vaccine, where an antigen for the vaccine is a fusion protein of Mhp, and has an amino acid sequence shown in SEQ ID NO: 1.

In the present disclosure, a concentration of the antigen in the vaccine is 50 μg/mL to 100 μg/mL.

In a preferred technical solution, the concentration of the antigen in the vaccine is 75 μg/mL to 85 μg/mL.

In the present disclosure, a dosage form of the vaccine is a water-in-oil-in-water form. In the present disclosure, an adjuvant in the vaccine is ISA201VG.

In the present disclosure, a preparation method of the fusion protein is as follows: inserting a coding gene for the fusion protein of Mhp into an expression vector, introducing into Escherichia coli, and inducing an expression to produce the fusion protein, where the coding gene for the fusion protein is shown in SEQ ID NO: 2.

The present disclosure also provides a preparation method of the vaccine, including the following steps:
(1) preparing an aqueous phase including the fusion protein; and
(2) mixing an oil phase with the aqueous phase according to a volume ratio of 1:(0.8-1.2), and emulsifying to produce the vaccine.

In the present disclosure, the oil phase is ISA201VG.

In the present disclosure, a concentration of the fusion protein in the aqueous phase is 100 μg/mL to 200 μg/mL.

In a preferred technical solution, the concentration of the fusion protein in the aqueous phase is 150 μg/mL to 170 μg/mL.

Beneficial effects: The fusion protein in the Mhp subunit vaccine of the present disclosure has a synergistic effect. The Mhp subunit vaccine can effectively prevent the infection of Mhp, with an efficiency of 80% or more and an immunization duration of two years. The Mhp subunit vaccine is safer and cheaper than the traditional vaccines.

### BRIEF DESCRIPTION OF THE DRAWINGS

FIGS. 1A-1B show sodium dodecyl sulfate-polyacrylamide gel electrophoresis (SDS-PAGE) electropherograms for the identification of a recombinant protein A after soluble expression and purification, where FIG. 1A shows an SDS-PAGE electropherogram for the identification after soluble expression, where lane 1: cells before induction, lane 2: cells after induction, lane 3: cell lysate supernatant 2 after induction, and lane 4: cell lysate precipitate after induction; and FIG. 1B shows an SDS-PAGE electropherogram of the recombinant protein A after purification, where the left lane is for the molecular weight marker and the right lane is for the purified protein A, with an asterisk marking a target band;

FIG. 2 shows levels of IgG antibodies in serum of pigs of each group that are detected by enzyme-linked immunosorbent assay (ELISA), where an x-coordinate represents a time after the first immunization; DO represents the day before the first immunization, D14 represents day 14 after the first immunization, and so on; in FIG. 2, A, B, C, D, E, and the existing antigen each represent a protein immunization-challenge group; and in FIG. 2, the “challenge group” represents a challenge control group and phosphate buffered saline (PBS) represents a blank control group;

FIG. 3 shows levels of antibodies in serum of pigs of each immunization group;

FIG. 4 shows test results of proliferation responses of peripheral blood mononuclear cells (PBMCs) to each antigen in pigs immunized with a corresponding vaccine, where in FIG. 4, A, B, C, D, E, and the existing antigen each represent a protein immunization-challenge group; PBS represents a blank control group; * indicates a significant difference compared with the blank control group (PBS) (P<0.05); and ** indicates an extremely-significant difference compared with the blank control group (P<0.01);

FIG. 5 shows secretions of interleukin-17 (IL-17) by PBMCs in pigs immunized with a corresponding vaccine under the stimulation of a corresponding antigen, where in FIG. 5, A, B, C, D, E, and the existing antigen each represent a protein immunization-challenge group; PBS represents a blank control group; * indicates a significant difference compared with the blank control group (PBS), P<0.05; ** indicates an extremely-significant difference compared with the blank control group (PBS), P<0.01; and *** indicates a further extremely-significant difference compared with the blank control group (PBS), P<0.001; and

FIG. 6 shows nucleic acid contents of Mhp in lung tissues that are detected by quantitative polymerase chain reaction (qPCR), where in FIG. 6, A, B, C, D, E, and the existing antigen each represent a protein immunization-challenge group; PBS represents a blank control group; ns indicates an insignificant difference; and ** indicates an extremely-significant difference compared with a challenge control group, P<0.01.

### DETAILED DESCRIPTION OF THE EMBODIMENTS

### Example 1 Preparation of Fusion Proteins of Mhp

In order to identify an antigen with an excellent immunization effect for an Mhp subunit vaccine, epitopes were selected from numerous antigen proteins by an omics-based method, and various polypeptides and proteins were designed. Amino acid sequences and coding gene sequences for some polypeptides and proteins were listed in Table 1. According to test results, only a fusion protein A (abbreviated as protein A) exhibited a prominent immunization effect. For the protein A, an amino acid sequence was shown in SEQ ID NO: 1 and a coding gene was shown in SEQ ID NO: 2.

The gene for the protein A was synthesized by a gene company and cloned into a pET-21a vector to produce an expression plasmid pET-21a-A. The expression plasmid was transformed into a BL21 (DE3) strain. Specific steps were as follows: BL21 (DE3) competent cells were taken out from a −80° C. freezer and immediately placed on ice for thawing. 1 μL of the expression plasmid pET-21a-A was added to the competent cells. A resulting system was incubated on ice for 20 min to 30 min, then immediately placed in a 42° C. water bath for 90 s, and then cooled on ice for 2 min to 3 min. 1 mL of an LB medium was added, and culturing was allowed in a 37° C. shaker at 100 rpm for 50 min to 60 min. 100 μL of a culture was pipetted and evenly coated on an ampicillin-resistant solid LB plate. The plate was inverted and incubated in a 37° C. incubator for 16 h. Single Escherichia coli colonies were picked, 1 mL of an ampicillin-resistant LB liquid medium was added, and culturing was allowed at 37° C. and 180 rpm for 5 h to 6 h. A plasmid was extracted and sequenced. A colony with a correct sequence was selected as a recombinant strain expressing the protein A.

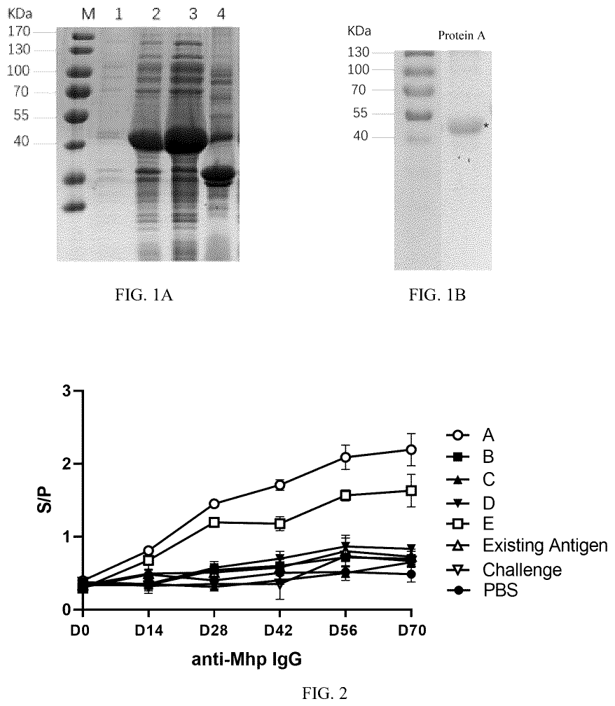

Preparation of the protein A: The recombinant strain expressing the protein A was inoculated into an LB medium including 100 mg/L of ampicillin, and culturing was allowed in a shaker at 37° C. and 180 rpm. When OD₆₀₀ reached 0.6 to 0.8, isopropyl-beta-D-thiogalactopyranoside (IPTG) was added at a final concentration of 1 mM, and an induction culture was allowed for 20 h at 24° C. and 150 rpm. Centrifugation was conducted at 6,000 g for 6 min to collect a cell pellet. A base buffer (a Tris-HCl buffer at a concentration of 30 mM and a pH of 8.0 that included 300 mM of NaCl and glycerol in a volume percentage of 2%) and phenylmethanesulfonyl fluoride (PMSF) at a final concentration of 0.1 mM were added to the cell pellet, and thorough suspending was allowed through pipetting up and down. The obvious clumps should be avoided. Ultrasonic disruption was conducted at 4° C. and a power of 65%, where working was continued for 2 s and stopped for 8 s, and a total ultrasonic time was 60 min. A solution produced after the ultrasonic disruption was centrifuged at 10,500 rpm and 4° C. for 30 min to produce lysate supernatant 1 and a lysate precipitate. The lysate supernatant 1 was centrifuged with an ultracentrifuge at 100,000 g and 4° C. for 40 min, and a resulting supernatant was collected as lysate supernatant 2 and filtered by a filter with a pore size of 0.22 μm. SDS-PAGE was conducted for the cells after induction, the lysate supernatant 2 after induction, and the lysate precipitate after induction, and results were shown in FIG. 1A. It could be known that the soluble expression of the protein A was achieved.

According to the conventional method, the lysate supernatant 2 was purified with a nickel column. A buffer for the purified protein A was replaced with PBS (pH 7.4) at a concentration of 0.01 M by an ultrafiltration tube, and concentration was conducted. As shown in FIG. 1B, the purified protein A had a high purity.

According to the preparation method for the protein A, coding genes for the existing antigen and proteins B, C, D, and E each were cloned into a pET-21a vector to produce expression plasmids, and the expression plasmids each were transformed into a BL21 (DE3) strain to produce expression strains for the proteins, respectively. After IPTG induction and purification, the existing antigen and proteins B, C, D, and E were obtained, which were provided to prove the immunization effect of the protein A.

TABLE 1
Amino acid and gene sequence numbers for the proteins

| Protein | Existing | | | | | |
|---|---|---|---|---|---|---|
| name | antigen | Protein B: | Protein C | Protein D | Protein E | Protein A |
| Amino acid | SEQ ID | SEQ ID | SEQ ID | SEQ ID | SEQ ID | SEQ ID |
| sequence number | NO: 11 | NO: 3 | NO: 5 | NO: 7 | NO: 9 | NO: 1 |
| Gene sequence | SEQ ID | SEQ ID | SEQ ID | SEQ ID | SEQ ID | SEQ ID |
| number | NO: 12 | NO: 4 | NO: 6 | NO: 8 | NO: 10 | NO: 2 |

### Example 2 Evaluation Test of an Immunoprotection Effect of the Protein a for Pigs

In order to investigate the immunoprotection effect of the protein A prepared in Example 1, Mhp subunit vaccines were prepared, including a protein A vaccine and control vaccines, and a piglet immunization and challenge test was carried out. The control vaccines included a protein B vaccine, a protein C vaccine, a protein D vaccine, a protein E vaccine, and an existing antigen vaccine.

A preparation method for each vaccine was as follows:

A preparation method for the protein A vaccine was as follows: A 160 μg/mL protein A solution was mixed with an equal volume of an ISA201VG adjuvant (Seppic, France), and emulsification was conducted with a homogenizer to produce a water-in-oil-in-water emulsion including 80 μg/mL of the antigen to produce the protein A vaccine. A solvent in the protein A solution was PBS at a concentration of 0.01 M and a pH of 7.4.

Preparation methods for the protein B vaccine, the protein C vaccine, the protein D vaccine, the protein E vaccine, and the existing antigen vaccine were the same as the preparation method for the protein A vaccine, except that a 160 μg/mL protein B solution, a 160 μg/mL protein C solution, a 160 μg/mL protein D solution, a 160 μg/mL protein E solution, and a 160 μg/mL existing antigen solution were adopted instead of the 160 μg/mL protein A solution, respectively. Solvents in the protein B solution, the protein C solution, the protein D solution, the protein E solution, and the existing antigen solution each were PBS at a concentration of 0.01 M and a pH of 7.4.

A preparation method for each protein was shown in Example 1.

Antigens and contents thereof in the above-mentioned Mhp subunit vaccines were shown in Table 2.

TABLE 2
Antigens in the Mhp subunit vaccines and piglet groups

| | | | Antigen content |
|---|---|---|---|
| | Vaccine to be | | in a vaccine |
| Piglet group | inoculated | Vaccine antigen | (μg/mL) |
| Protein B immunization- | Protein B vaccine | Protein B | 80 |
| challenge group | | | |
| Protein C immunization- | Protein C vaccine | Protein C | 80 |
| challenge group | | | |
| Protein D immunization- | Protein D vaccine | Protein D | 80 |
| challenge group | | | |
| Protein A immunization- | Protein A vaccine | Protein A | 80 |
| challenge group | | | |
| Protein E immunization- | Protein E vaccine | Protein E | 80 |
| challenge group | | | |
| Existing antigen | Existing antigen | Existing antigen (with an | 80 |
| immunization-challenge | vaccine | amino acid sequence | |
| group | | shown in SEQ ID NO: 11) | |
| Blank control group | PBS | — | 0 |
| Challenge control group | PBS | — | 0 |
| Protein A immunization | Protein A vaccine | Protein A | 80 |
| group | | | |
| Existing antigen | Existing antigen | Existing antigen (with an | 80 |
| immunization group | vaccine | amino acid sequence | |
| | | shown in SEQ ID NO: 11) | |
| Protein E immunization | Protein E vaccine | Protein E | 80 |
| group | | | |

### 1. Animal Grouping and Immunization Program

47 ternary crossbred pigs that were 5 to 15 days old, were negative for anti-pseudorabies virus and anti-PRRSV antibodies and negative for African swine fever virus, Haemophilus parasuis, SS, Mycoplasma hyorhinis, and Mhp antigens, and was not immunized with any vaccine were randomly divided into 11 groups, including a protein B immunization-challenge group, a protein C immunization-challenge group, a protein D immunization-challenge group, a protein E immunization-challenge group, a protein A immunization-challenge group, an existing antigen (a polypeptide disclosed in the literature) immunization-challenge group, a challenge control group, a blank control group, a protein A immunization group, an existing antigen immunization group, and a protein E immunization group.

There were 5 pigs in each immunization-challenge group and the challenge control group, and there were 3 pigs in each immunization group and the blank control group. Each ternary crossbred pig in each immunization group and each immunization-challenge group was intramuscularly injected with 1 mL of a corresponding vaccine at the right triangle neck. 2 weeks later, the booster immunization was conducted once with the same immunization mode and dose as the first immunization. 2 immunizations were conducted in total. The blank control group and the challenge control group each were injected with PBS at 0.01 M and pH 7.4 at the same dose in the same way.

### 2. Challenge and Calculation of Challenge Protection Rates

6 weeks after the first immunization, each immunization-challenge group and the challenge control group were challenged, and each immunization group and the blank control group were injected with PBS at the same dose in the same way. A virulent strain AV747 of Mhp (published in the Chinese patent ZL201410287236.8) was cultured until a late logarithmic growth stage, and cells were resuspended with sterilized 0.01 M and pH 7.4 PBS to 109.5 CCU/5 mL for challenge. The challenge control group and each immunization-challenge group were challenged in the following way: The strain AV747 was inoculated through transtracheal injection, with 5 mL for each pig (109.5 CCU in total). 24 h later, the challenge was repeated once at the same dose in the same way. Transtracheal injection: A pig was fixed by a slanted hold frame with a “head-up and feet-down” supine position, and the virulent virus was inoculated through a tracheal route. After the inoculation, the pig must be fixed and kept still for at least 5 s, such that the challenge was completed. 28 d (D70) after the first challenge, each immunization-challenge group, the challenge control group, and the blank control group were subjected to necropsy. A lung lesion degree was determined by a 28-point scoring method, and a challenge protection rate was calculated. “D70” indicated 70 d after the first immunization, and so on.

Statistics and evaluation of challenge protection rates: D70 experimental pigs were sacrificed and evaluated according to the evaluation method for lung lesions: 28-point scoring evaluation method [Madec F, Kobish M (1982) Gross lung lesions of pigs at slaughter. Journées de la Recherche Porcine 14:405-412 (in French)]. Lesion percentages of left apical lobes, left cardiac lobes, left diaphragmatic lobes, right apical lobes, right cardiac lobes, right diaphragmatic lobes, and accessory lobes of ternary crossbred pigs in each of the immunization-challenge groups, the challenge control group, and the blank control group were recorded. A lesion percentage of 0% to 12.5% was denoted as 0.5, a lesion percentage of 12.5% to 25% was denoted as 1, a lesion percentage of 25% to 37.5% was denoted as 1.5, a lesion percentage of 37.5% to 50% was denoted as 2, a lesion percentage of 50% to 62.5% was denoted as 2.5, a lesion percentage of 62.5% to 75% was denoted as 3, a lesion percentage of 75% to 87.5% was denoted as 3.5, and a lesion percentage of 87.5% to 100% was denoted as 4. A lung lesion index of each experimental pig was counted (published in Madec F, Kobish M (1982) Gross lung lesions of pigs at slaughter. Journées de la Recherche Porcine 14:405-412 (in French)), and an average lung lesion index was calculated for the challenge control group, the immunization-challenge groups, and the blank control group. Based on a lung lesion index, a lung lesion index reduction rate was calculated according to the following formula: lung lesion index reduction rate=(average lung lesion index of the challenge control group-average lung lesion index of an immunization-challenge group)/(average lung lesion index of the challenge control group-average lung lesion index of the blank control group)×100%. According to the formula for calculating a vaccine efficiency in the third edition of “Principles of Epidemiology in Public Health Practice” released by the Centers for Disease Control and Prevention (CDC) of the United States: vaccine efficiency=(number of diseased cases in a non-immunization group-number of diseased cases in an immunization-challenge group)/number of diseased cases in the non-immunization group, a vaccine efficiency was calculated. The non-immunization group was the challenge control group. Lung lesion index reduction rates and vaccine efficiencies were shown in Table 3.

TABLE 3
Indexes for evaluating immunoprotection effects of vaccines lesion

| | Lung | | | Lung lesion |
|---|---|---|---|---|
| | lesion | Average lung | Vaccine | index reduction |
| Group | index | lesion index | efficiency (%) | rate (%) |
| Protein B immunization- | 0 | 3.8 | 20 | 64.8 |
| challenge group | 6 | | | |
| | 6 | | | |
| | 3 | | | |
| | 4 | | | |
| Protein C immunization- | 0 | 4.2 | 20 | 61.1 |
| challenge group | 8 | | | |
| | 6 | | | |
| | 5 | | | |
| | 2 | | | |
| Protein D immunization- | 7 | 4 | 20 | 63.0 |
| challenge group | 0 | | | |
| | 4 | | | |
| | 4 | | | |
| | 5 | | | |
| Protein A immunization- | 0 | 1.2 | 80 | 88.9 |
| challenge group | 0 | | | |
| | 6 | | | |
| | 0 | | | |
| | 0 | | | |
| Protein E immunization- | 1 | 3.8 | 20 | 64.8 |
| challenge group | 6 | | | |
| | 6 | | | |
| | 6 | | | |
| | 0 | | | |
| Existing antigen | 0 | 4.2 | 40 | 61.1 |
| immunization-challenge | 9 | | | |
| group | 7 | | | |
| | 5 | | | |
| | 0 | | | |
| Challenge control group | 9 | 10.8 | — | — |
| | 9 | | | |
| | 10 | | | |
| | 12 | | | |
| | 14 | | | |
| Blank control group | 0 | 0 | — | — |
| | 0 | | | |
| | 0 | | | |

Results: As shown in Table 3, according to the formula for calculating a vaccine efficiency in the third edition of “Principles of Epidemiology in Public Health Practice” released by the Centers for Disease Control and Prevention (CDC) of the United States, a vaccine efficiency of the Mhp protein B vaccine after inoculation was (5-4)/5=20%. Based on a lung lesion index, a lung lesion index reduction rate was calculated to be [(9+9+10+12+14)/5−(6+6+7)/5]/[(9+9+10+12+14)/5]=64.8%. Similarly, the protein C vaccine had an efficiency of 20% and a lung lesion index reduction rate of 61.1%. The protein D vaccine had an efficiency of 20% and a lung lesion index reduction rate of 63.0%. The protein A vaccine had an efficiency of 80% and a lung lesion index reduction rate of 88.9%. The protein E vaccine had an efficiency of 20% and a lung lesion index reduction rate of 64.8%. The existing antigen vaccine had an efficiency of 40% and a lung lesion index reduction rate of 61.1%. The protein A vaccine had an efficiency of 80% and a lung lesion index reduction rate of 88.9%, and exhibited the highest protection rate, indicating that the protein A vaccine had the optimal immunoprotection effect.

### 3. Detection of IgG Levels in Serum by ELISA

Blood was collected from the immunization-challenge groups, the challenge control group, and the blank control group before immunization (DO), 14 d after the first immunization (D14), 28 d after the first immunization (D28), 28 d after the second immunization (D42), 7 d after the first challenge (D49), 14 d after the first challenge (D56), and 28 d after the first challenge (D70), and scrum was isolated. In each immunization group, blood was collected every 3 months and subjected to serum isolation until 2 years after the first immunization (D720). A scrum IgG level was detected by an Mhp antibody test kit (purchased from IDEXX of the United States, product No. 99-06733) according to the instructions of the kit, and S/P was calculated. When S/P >0.4, it was positive.

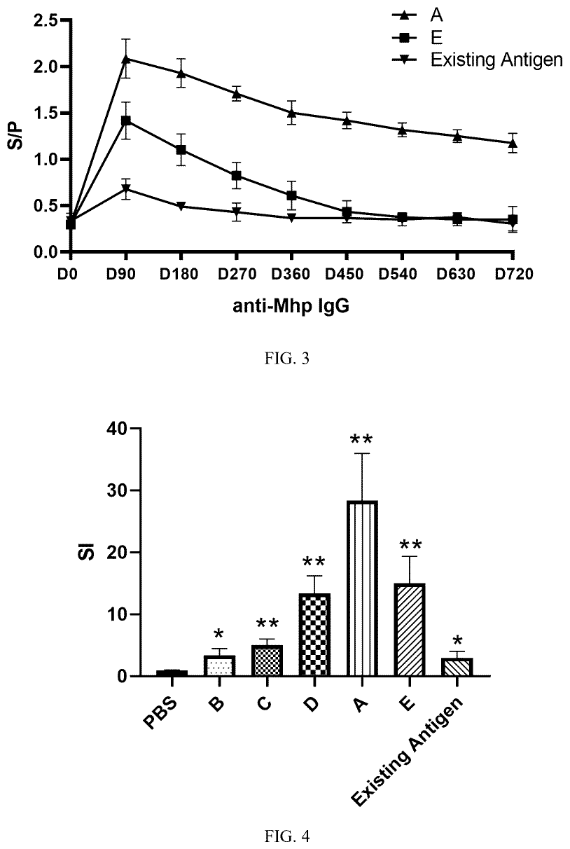

Results: As shown in FIG. 2, each immunization-challenge group could produce a specified IgG level, and the protein A induced the highest IgG level in serum of pigs. FIG. 3 shows test results of an immunization duration of the protein A immunization group. It can be seen that the ternary crossbred pigs can still maintain a high IgG level 2 years after the immunization with the protein A vaccine, and an immunization duration reaches 2 years. The protein A immunization group has a longer immunization duration than the existing antigen and protein E immunization groups that each had an immunization duration of less than one year.

### 4. Detection of Proliferation of PBMCs in Pigs

Proliferation responses of PBMCs in pigs immunized with different vaccines (the immunization-challenge groups and blank control group) were detected, and specific cellular immune responses were evaluated.

After the immunization (D42) with a vaccine in each immunization-challenge group, porcine PBMCs were extracted using a porcine PBMC isolation kit (Beijing Solarbio Science. & Technology Co., Ltd., P4420), and stimulated with an antigen for the immunization to investigate the influence of each antigen on the specific proliferation response of porcine PBMCs. A specific process was as follows: 50 μL of a suspension including 1× 10⁵ PBMCs was inoculated in each well of a 96-well plate. To each of wells with PBMCs of the protein B immunization-challenge group, the protein C immunization-challenge group, the protein D immunization-challenge group, the protein A immunization-challenge group, the protein E immunization-challenge group, and the existing antigen immunization-challenge group, respectively, 2.5 μg of an antigen in a corresponding vaccine was added in a volume of 50 μL for stimulation. To each well with porcine PBMCs of the blank control group, 50 μL of PBS was added for stimulation. Positive control wells and negative control wells were set for PBMCs in pigs immunized with each vaccine. In the positive control wells, concanavalin at a concentration of 25 μg/mL was added instead of a protein solution for stimulation. In the negative control wells, PBS was added for stimulation. Incubation was conducted for 72 h. Then a CCK-8 solution was added to each well, incubation was conducted for 4 h in a 37° C. and 5% CO₂ incubator, and an OD₄₅₀ value was detected by a microplate reader. Wells with only a medium added were adopted as blank control wells. Stimulation index (SI)=(OD value of an antigen stimulation well−OD value of a blank control well)/(OD value of a negative control well−OD value of a blank control well).

Results: The positive and negative controls both were established. Test results of antigen stimulation wells were shown in Table 4 and FIG. 4. The protein B, protein C, and protein D alone as an antigen all had a significantly higher SI value than the blank control group, indicating that these three proteins all could stimulate the proliferation of PBMCs and enhance the cellular immune response. The protein E could enhance the response of immune cells. However, the protein A had a significantly-higher SI value than other proteins, and led to the strongest effect of enhancing the cellular immune response.

TABLE 4
SI corresponding to the stimulation of each antigen corresponding

| Vaccine corresponding to blood | | |
|---|---|---|
| mononuclear cells in a sample well | Antigen for stimulation | SI |
| Protein B vaccine | Protein B | 3.3 |
| Protein C vaccine | Protein C | 5 |
| Protein D vaccine | Protein D | 13.3 |
| Protein A vaccine | Protein A | 28.3 |
| Protein E vaccine | Protein E | 15 |
| Existing antigen vaccine | Existing antigen reported in the | 3 |
| | literature (with an amino acid | |
| | sequence shown in SEQ ID NO: 11) | |
| Blank control group | PBS | 1 |

### 5. Detection of Induction and Secretion of Specific IL-17 of PBMCs in Pigs by ELISA

After the immunization (D42) with a vaccine in each immunization-challenge group, porcine PBMCs were extracted using a porcine PBMC isolation kit (Beijing Solarbio Science. & Technology Co., Ltd., P4420), and stimulated with an antigen for the immunization to investigate the influence of each antigen on the secretion of specific IL-17 of PBMCs in pigs. A specific process was as follows: 1×10⁵ mononuclear cells were inoculated with a volume of 50 μL in each well of a 96-well plate. To each of wells with porcine PBMCs of the protein B immunization-challenge group, the protein C immunization-challenge group, the protein D immunization-challenge group, the protein A immunization-challenge group, the protein E immunization-challenge group, and the existing antigen immunization-challenge group, respectively, 2.5 μg of an antigen in a corresponding vaccine was added in a volume of 50 μL. To each well with porcine PBMCs of the blank control group, 50 μL of PBS was added for stimulation. Positive control wells and negative control wells were set for PBMCs in pigs immunized with each vaccine. In the positive control wells, concanavalin at a concentration of 25 μg/mL was added instead of a protein solution for stimulation. In the negative control wells, PBS was added instead of a protein solution for stimulation. Incubation was conducted for 72 h. A cell culture supernatant of each well was collected and tested for an IL-17 content by a porcine IL 17A test kit (Beijing Solarbio Science. & Technology Co., Ltd., SEKP-0173).

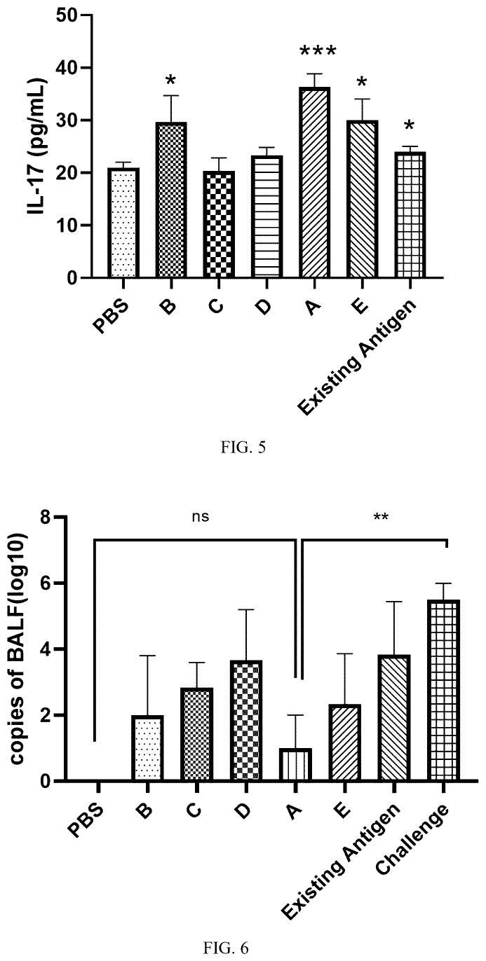

Results: The positive and negative controls both were established. Results of the stimulation of PBMCs in immunized pigs with antigens were shown in Table 5 and FIG. 5. When the protein B, protein C, and protein D each were adopted alone as an antigen for stimulation, an IL-17 level secreted by PBMCs in stimulated pigs was significantly higher than a corresponding IL-17 level of the blank control group. However, the protein A exhibited a significantly-stronger stimulation ability for IL-17 than other antigens, indicating the optimal stimulation ability for cellular immunity.

TABLE 5
Secretions of specific IL-17 by porcine PBMCs for different proteins

| Vaccine corresponding to | | Secretion of specific IL-17 by |
|---|---|---|
| PBMCs in a sample well | Antigen for stimulation | PBMCs (pg/mL) |
| Protein B vaccine | Protein B | 29.7 |
| Protein C vaccine | Protein C | 20.3 |
| Protein D vaccine | Protein D | 23.3 |
| Protein A vaccine | Protein A | 36.3 |
| Protein E vaccine | Protein E | 30 |
| Existing antigen vaccine | Existing antigen (with an | 24 |
| | amino acid sequence shown in | |
| | SEQ ID NO: 11) | |
| Blank control group | PBS | 21 |

6. Detection of Pathogen Loads in Porcine Tissues by qPCR

28 d after the challenge (D70), a bronchoalveolar lavage fluid (BALF) of a diseased pig was collected. Resected lung lobes and other damaged areas of lungs were clamped with hemostats, and 50 mL of sterile PBS at 0.01 M and pH 7.4 was drawn with a syringe and perfused into the lungs along a trachea. Each lung lobe was gently massaged and patted. A liquid poured out after 2 min was BALF. 1 mL of BALF was taken and centrifuged at 10,000 r/min for 20 min. A resulting precipitate was collected and subjected to DNA extraction with a nucleic acid extraction kit. Pathogenic nucleic acid contents of the immunization-challenge groups, the challenge control group, and the blank control group were detected by qPCR. A specific process could refer to the following: Wu Yuzi et al., Establishment and Use of Method for Detecting Mycoplasma hyopneumoniae (Mhp) P97 based on TaqMan-BHQ fluorescent qPCR, Veterinary Science in China, 2012, 42 (12): 1268-1272.

As shown in FIG. 6, nucleic acid contents of Mhp in BALFs of the immunization-challenge groups that were inoculated with the corresponding Mhp subunit vaccines and challenged all were lower than a corresponding nucleic acid content of the challenge control group to varying degrees. A nucleic acid content in BALF of the protein A immunization-challenge group was significantly lower than a corresponding nucleic acid content of the challenge control group, was less than 50% of a corresponding nucleic acid content of the protein B immunization-challenge group, and was not significantly different from the blank control group, indicating that the protein A could significantly reduce the tissue load of Mhp.

What is claimed is:

1. A Mycoplasma hyopneumoniae (Mhp) subunit vaccine, wherein an antigen for the Mhp subunit vaccine is a fusion protein of Mhp, and has the amino acid sequence shown in SEQ ID NO: 1.

2. The Mhp subunit vaccine according to claim 1, wherein a concentration of the antigen in the Mhp subunit vaccine is 50 μg/mL to 100 μg/mL.

3. The Mhp subunit vaccine according to claim 2, wherein the concentration of the antigen in the Mhp subunit vaccine is 75 μg/mL to 85 μg/mL.

4. The Mhp subunit vaccine according to claim 1, wherein a dosage form of the Mhp subunit vaccine is a water-in-oil-in-water form.

5. The Mhp subunit vaccine according to claim 4, wherein an adjuvant in the Mhp subunit vaccine is ISA201VG.

6. The Mhp subunit vaccine according to claim 5, wherein a preparation method of the fusion protein is as follows: inserting a coding gene for the fusion protein of Mhp into an expression vector, introducing into Escherichia coli, and inducing an expression to produce the fusion protein, wherein the coding gene for the fusion protein is shown in SEQ ID NO: 2.

7. A preparation method of the Mhp subunit vaccine according to claim 1, comprising the following steps:
(1) preparing an aqueous phase comprising the fusion protein; and
(2) mixing an oil phase with the aqueous phase according to a volume ratio of 1:(0.8-1.2), and emulsifying to produce the Mhp subunit vaccine.

8. The preparation method according to claim 6, wherein the oil phase is ISA201VG.

9. The preparation method according to claim 7, wherein a concentration of the fusion protein in the aqueous phase is 100 μg/mL to 200 μg/mL.

10. The preparation method according to claim 9, wherein the concentration of the fusion protein in the aqueous phase is 150 μg/mL to 170 μg/mL.

11. The Mhp subunit vaccine according to claim 2, wherein a dosage form of the Mhp subunit vaccine is a water-in-oil-in-water form.

12. The Mhp subunit vaccine according to claim 3, wherein a dosage form of the Mhp subunit vaccine is a water-in-oil-in-water form.

13. The Mhp subunit vaccine according to claim 11, wherein an adjuvant in the Mhp subunit vaccine is ISA201VG.

14. The Mhp subunit vaccine according to claim 12, wherein an adjuvant in the Mhp subunit vaccine is ISA201VG.

15. The Mhp subunit vaccine according to claim 13, wherein a preparation method of the fusion protein is as follows: inserting a coding gene for the fusion protein of Mhp into an expression vector, introducing into Escherichia coli, and inducing an expression to produce the fusion protein, wherein the coding gene for the fusion protein is shown in SEQ ID NO: 2.

16. The Mhp subunit vaccine according to claim 14, wherein a preparation method of the fusion protein is as follows: inserting a coding gene for the fusion protein of Mhp into an expression vector, introducing into Escherichia coli, and inducing an expression to produce the fusion protein, wherein the coding gene for the fusion protein is shown in SEQ ID NO: 2.
